(19) United States
(12) Patent Application Publication
(10) Pub. No.: US 2008/0294189 A1
(43) Pub. Date: Nov. 27, 2008

(54) VEIN FILTER

(76) Inventors: Fransiscus L. Moll, Bosch En Duin (NL); Menno Kalmann, Elspeet (NL); Peter W.J. Hinchliffe, Campbell Hall, NY (US); Adam I. Lehman, Northford, CT (US)

Correspondence Address: Edwards Angell Palmer & Dodge LLP, P.O. Box 55874, Boston, MA, 02205, US

(21) Appl. No.: 12/126,689

(22) Filed: May 23, 2008

Related U.S. Application Data
(60) Provisional application No. 60/931,403, filed on May 23, 2007.
(60) Provisional application No. 61/008,682, filed on Dec. 21, 2007.

(51) Int. Cl. A61M 29/02

(52) U.S. Cl. 606/200

(57) ABSTRACT

A vein filter includes a plurality of circumferentially spaced apart elongated legs having opposed proximal and distal leg portions. Adjacent distal leg portions are connected to one another by bridging structure and the legs are adapted and configured for movement between a first position and a second position. In the first position, the proximal leg portions are substantially parallel to one another. In the second position, the proximal leg portions are circumferentially divergent from one another to anchor the filter within a blood vessel. Methods of placing or relocating a vein filter in a vein utilize a catheter balloon to move the vein filter legs between first and second positions. Kits include a vein filter, a catheter balloon for moving the legs of the vein filter, and a delivery sheath for housing the vein filter for delivery within a blood vessel.

### CROSS-REFERENCE TO RELATED APPLICATIONS

This application claims benefit of priority to U.S. Provisional Application Ser. No. 60/931,403 filed May 23, 2007 and to U.S. Provisional Application Ser. No. 61/008,682 filed Dec. 21, 2007, the disclosures of which are incorporated by reference herein in their entireties.

### BACKGROUND OF THE INVENTION

1. Field of the Invention

This application relates to vessel filters for filtering blood clots and more particularly to vein filters that can be relocated or removed from the vessel after a period of time.

2. Description of Related Art

Filters can be used within blood vessels to reduce the risk of blood clots (emboli) migrating to critical portions of the circulatory system. Without preventative measures or treatment, blood clots can constitute a serious health threat and can even be fatal. Vein filters are frequently placed in the inferior vena cava either through the femoral or jugular vein to prevent blood clots formed in the lower body during surgery or trauma from entering the pulmonary artery and causing pulmonary embolus.

As described in U.S. Patent Application No. 2003/0139765 to Patel et al., a vein filter must be removed or relocated within about fourteen days of being placed in a vein unless it is intended for the vein filter to remain permanently in the vein. It takes roughly fourteen days for a significant amount of endothelial cells to thicken around the contacts of a vein filter on a blood vessel wall. Significant risks arise when attempting to remove a vein filter that has become so integrated with the vein wall, including lacerating/rupturing the vessel wall, or risking thrombosis, occlusion, or other complications. It can therefore be very important that a vein filter be readily removable to avoid unwanted permanence of the vein filter.

For various reasons, approximately 20%-30% of the time vein filters are not optimally placed on the first attempt. A typical vein filter that is not in the proper location must be removed, which in the case of an improperly placed filter in the inferior vena cava entering from the femoral vein, involves gaining access for retrieval through the jugular vein. Moreover, once retrieved, typical vein filters must be discarded, since they are not configured for reuse, and a new procedure using a new vein filter must then be attempted. Thus with previously known vein filters, initial misplacement of the filter occurs frequently and can be very costly. Due to the cost and difficulty of repositioning typical vein filters, physicians may feel compelled to accept the initial placement of filters that are not optimally placed if the risks of leaving the filter are outweighed by the costs and risks of replacement with a better placed filter.

While various approaches have been taken to make vein filters easier to move within a blood vessel, there remains a continued need for vein filters that can be more readily repositioned within a blood vessel. The present invention provides a solution for this problem.

### SUMMARY OF THE INVENTION

The subject invention is directed to a new and useful vein filter for blood vessels. The vein filter includes a plurality of circumferentially spaced apart elongated legs having opposed proximal and distal leg portions. Adjacent distal leg portions are connected to one another by bridging structure. The legs are adapted and configured for movement between a first position wherein the proximal leg portions are substantially parallel to one another and a second position wherein the proximal leg portions are circumferentially divergent from one another to anchor the filter within a blood vessel.

In one aspect, the legs and/or bridging structure are biased to the second position to anchor the filter within a blood vessel. It is contemplated that the legs and bridging structure can include a shape memory material biasing the legs and bridging structure to the second position. The distal leg portions can define a receptacle configured and adapted to receive a guidewire and catheter balloon or other expanding member for expanding against the distal leg portions to place the legs in the first position for vascular introduction and/or relocation, and for contracting to allow the distal leg portions to converge to place the legs in the second position to anchor the filter within a blood vessel.

In another aspect, each of the proximal leg portions includes means for anchoring the filter to a blood vessel wall. It is contemplated that each of the proximal leg portions can include an enlarged section configured and adapted to engage a blood vessel wall to limit penetration thereof. The legs can be spaced substantially equally apart circumferentially. It is contemplated that there can be at least six legs, or any other suitable number of legs. Adjacent distal leg portions can be connected to one another by bridging structure that defines a pivot point for movement of each of the legs between the first and second positions. It is also envisioned that the legs and bridging structure can be a continuous integral structure. The vein filter can further include retrieval means connected to at least one distal leg portion, the retrieval means being configured and adapted to receive a retrieval device for removing the vein filter distally.

A kit is provided for placing a vein filter within a vein. The kit includes a vein filter having a plurality of circumferentially spaced apart elongated legs with opposed proximal and distal leg portions. Adjacent distal leg portions are connected to one another by bridging structure. The legs are adapted and configured for movement between a first position wherein the proximal leg portions are substantially parallel to one another and a second position wherein the proximal leg portions are circumferentially divergent from one another to anchor the filter within a blood vessel. The distal leg portions define a receptacle configured and adapted to receive a guidewire and catheter balloon. The kit further includes a catheter balloon configured and adapted to expand against the distal leg portions to place the legs of the filter in the first position for vascular introduction and/or repositioning, and to contract to allow the distal leg portions to converge to place the legs in the second position to anchor the filter within a blood vessel. The kit also includes a delivery sheath configured and adapted to house the vein filter, and if applicable the catheter balloon, during placement of the vein filter within a blood vessel.

In another aspect, it is contemplated that the legs of the vein filter can be dimensioned and adapted for placement within an inferior vena cava. The catheter balloon can include means for fluoroscopically determining the position of the catheter balloon with respect to the distal leg portions of the vein filter

A method is provided for placing a vein filter within a vein. The method includes providing a vein filter substantially as described above wherein the distal leg portions of the vein filter define a receptacle for receiving a catheter balloon for inflating against the distal leg portions of the vein filter to move the legs into the first position, and wherein the vein filter is housed within a delivery sheath with the legs in substantially the first position for placement within a blood vessel. The method further includes positioning a guidewire within a vein for guiding the vein filter and delivery sheath to a predetermined location within a vein and guiding the vein filter and delivery sheath into the predetermined location along the guidewire. A step is provided for withdrawing the delivery sheath from the vein filter to anchor the vein filter within the vein with the legs in the second position.

It is contemplated that the step of guiding can be accomplished using fluoroscopy to guide the vein filter to the predetermined location within the vein. The method can further include repositioning the vein filter by placing a deflated catheter balloon within the receptacle of the distal leg portions of the vein filter, inflating the catheter balloon to place the legs of the vein filter into substantially the first position, relocating the vein filter together with the catheter balloon, deflating the catheter balloon to place the legs of the vein filter into the second position, and removing the catheter balloon from the vein filter. It is also envisioned that the step of repositioning can include placing the deflated catheter balloon within the receptacle of the vein filter by introducing the catheter balloon over a guidewire through a jugular vein.

In another aspect, it is contemplated that the step of providing a vein filter can include providing a catheter balloon within the distal leg portions of the vein filter. The catheter balloon can be configured and adapted to expand against the distal leg portions of the vein filter to place the legs of the vein filter in the first position and to contract to place the legs of the vein filter in the second position. The step of withdrawing the delivery sheath from the vein filter can include contracting the catheter balloon or other expandable member to place the legs of the vein filter in the second position to anchor the vein filter within the vein.

These and other features of the systems and methods of the subject invention will become more readily apparent to those skilled in the art from the following detailed description of the preferred embodiments taken in conjunction with the drawings.

### BRIEF DESCRIPTION OF THE DRAWINGS

So that those skilled in the art to which the subject invention appertains will readily understand how to make and use the methods and devices of the subject invention without undue experimentation, preferred embodiments thereof will be described in detail herein below with reference to certain figures, wherein:

FIG. 1A is a perspective view of a first representative embodiment of a vein filter constructed in accordance with the present invention, showing the vein filter in the expanded (deployed) configuration;

FIG. 1B is a side view of the filter of FIG. 1A, showing the gap or receptacle for receiving a catheter balloon in the distal end of the filter;

FIG. 1C is a front (distal) view of the filter of FIG. 1A, showing the legs interconnected by bridging structure;

FIG. 2A is a perspective view of a further embodiment of a vein filter constructed in accordance with the present invention, showing the filter in the collapsed (delivery) configuration;

FIG. 2B is a perspective view of the filter of FIG. 2A, showing the filter in the expanded (deployed) configuration;

FIG. 2C is a perspective view of the filter of FIG. 2B, showing an upstream (proximal) end of the filter;

FIG. 3A is an enlarged perspective view of the distal region of the filter of FIG. 2B, showing a hook for retrieval of the filter;

FIG. 3B is an enlarged perspective view of a distal portion of another representative embodiment of a vein filter constructed in accordance with the present invention, showing the ends of the legs of the filter;

FIG. 3C is an enlarged view of a proximal portion of the filter of FIG. 2B, showing the hook (barb) for fixation of the filter within a blood vessel;

FIGS. 4-13 are perspective views illustrating a method of inserting a filter in a vein and of removing the filter from the vein in accordance with the present invention, a portion of the vein being cut away for ease of illustration, wherein

FIG. 4A is a perspective view of the filter of FIG. 1A and a catheter balloon contained within a delivery catheter or sheath, being inserted over a guidewire into the vessel, the balloon being in the inflated condition;

FIG. 4B is a close up perspective view of the filter of FIG. 1A, showing the positioning of the inflated balloon in the pivot region of the filter;

FIG. 5 is a perspective view of the filter of FIG. 1A and delivery sheath in the vessel at the desired position of deployment, showing the balloon in the inflated condition;

FIG. 6 is a perspective view of the filter of FIG. 1A, showing the delivery sheath being withdrawn to expose the filter;

FIG. 7A is a perspective view of the filter of FIG. 1A, showing the balloon deflated, enabling the filter to move to an expanded position wherein the filter hooks engage the vessel wall;

FIG. 7B is a close up perspective view of the distal region of the filter of FIG. 1A and deflated balloon of FIG. 7A, showing the distal ends of the filter legs in the deployment position;

FIG. 8 is a perspective view of the filter of FIG. 1A, showing retraction of the deflated balloon, delivery sheath and guidewire, leaving the filter in the vessel in the expanded position;

FIG. 9 is a perspective view of the filter of FIG. 1A, showing the filter in the expanded (deployed) position within the vessel;

FIG. 10 is a perspective view of the filter of FIG. 1A, showing reinsertion of the balloon and sheath over the guidewire into the vessel for withdrawal of the filter;

FIG. 11 is a perspective view of the filter of FIG. 1A, showing the balloon catheter inserted into the filter and the inflated balloon engaging the distal region of the filter to move the filter to the smaller profile collapsed configuration;

FIG. 12 is a perspective view of the filter of FIG. 1A, showing advancement of the delivery sheath over the filter and balloon;

FIG. 13 is a perspective view of the filter of FIG. 1A, showing the balloon catheter, delivery sheath and collapsed filter being removed from the vessel;

FIG. 14A is a close up perspective view of the filter of FIG. 1A, showing the hook region in the proximal portion of a leg of the filter;

FIG. 14B is a perspective view of a further embodiment of a hook region of a vein filter constructed in accordance with the present invention, showing a curved hook portion;

FIG. 14C is a perspective view of a further embodiment of a hook region of a vein filter constructed in accordance with the present invention, showing a right-angle hook portion;

FIG. 15 is a perspective view of a further embodiment of a hook region of a vein filter constructed in accordance with the present invention, showing a hook having a sharpened tip;

FIG. 16 is a perspective view of the filter of FIG. 1A, showing access to the filter by a balloon catheter via a jugular approach;

FIG. 17 is a perspective view of the filter of FIG. 1A and delivery sheath in the vessel at the desired position of deployment, showing use of the filter without a balloon catheter;

FIG. 18 is a perspective view of the filter of FIG. 1A and a delivery sheath in the vessel at the desired position of deployment, showing the delivery sheath partially withdrawn without using a balloon catheter for deployment;

FIG. 19 is a perspective view of the filter of FIG. 1A, showing the delivery sheath withdrawn from the filter, the filter legs being expanded against the vessel wall after deployment without the use of a balloon catheter;

FIG. 20A is a perspective view of an embodiment of a retractable barb region of a vein filter constructed in accordance with the present invention, showing a retractable barb attached to a living hinge defined by a notch in the top of the hinge;

FIG. 20B is a side elevation view of the retractable barb region of a vein filter of FIG. 20A, showing the barb in the retracted position;

FIG. 20C is a side elevation view of the retractable barb region of a vein filter of FIG. 20A, showing the barb in the extended position;

FIG. 21 is a perspective view of the another embodiment of a retractable barb portion of a vein filter constructed in accordance with the present invention, showing a barb in the retracted position with the proximal most end of the barb portion flush with the leg of the vein filter;

FIG. 22 is a side elevation view of another embodiment of a retractable barb portion of a vein filter constructed in accordance with the present invention, showing a barb connected to a living hinge defined by a notch on the bottom of the hinge; and

FIG. 23 is a perspective view of another embodiment of a retractable barb portion of a vein filter constructed in accordance with the present invention, showing a two-piece hinge for retraction and extension of the barb.

### DETAILED DESCRIPTION OF PREFERRED EMBODIMENTS

Reference will now be made to the drawings, wherein like reference numerals identify similar structural features or aspects of the subject invention. In accordance with the invention, a filter is provided for placement within the inferior vena cava to capture blood clots or other particles which could otherwise pass to the lungs. The vein filter is movable from an insertion (delivery) position, having a lower profile, to an expanded position with a larger profile. The low profile collapsed configuration allows for insertion and removal of the filter; the expanded position enables the hooks of the filter to engage the vessel wall to secure the filter. A balloon is utilized to move the filter to the reduced profile condition in the manner described below.

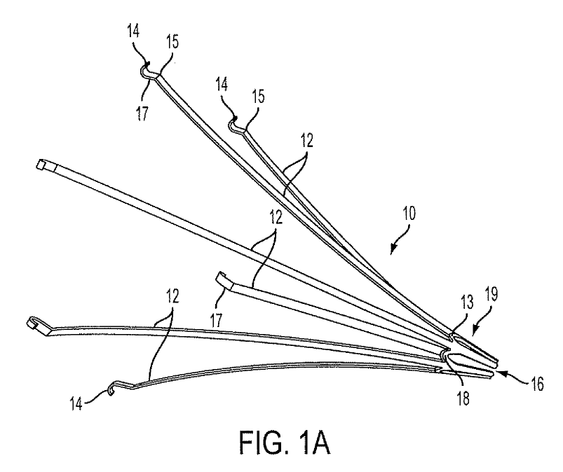

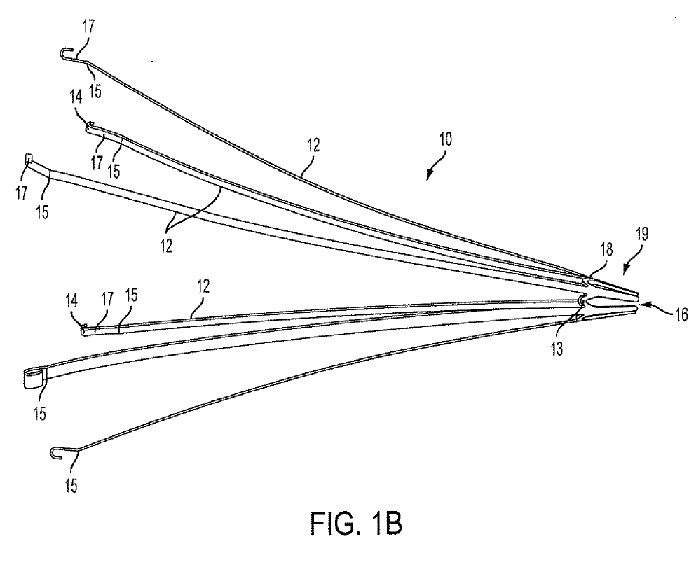

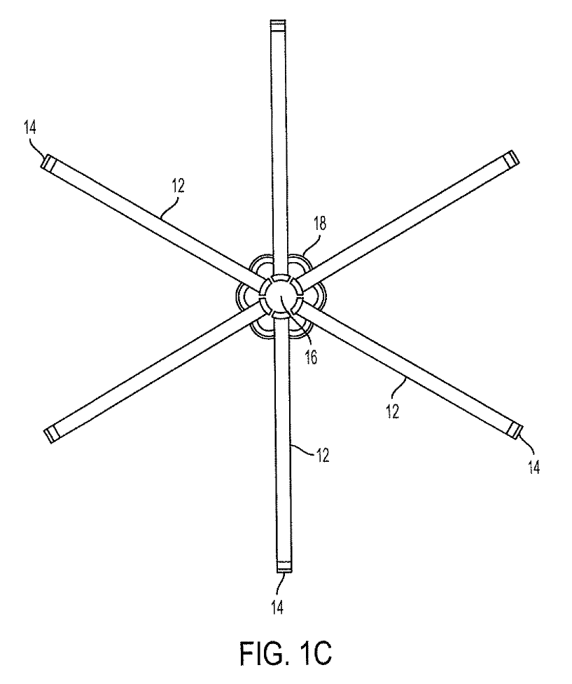

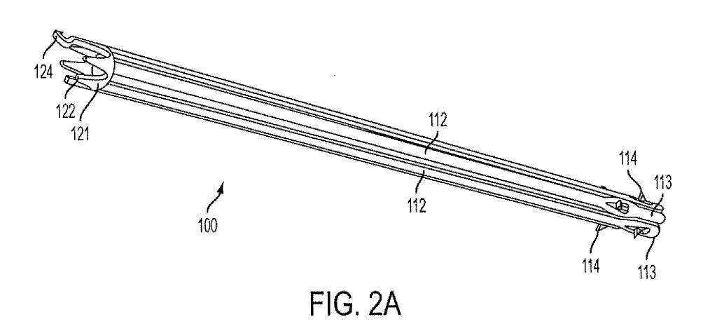

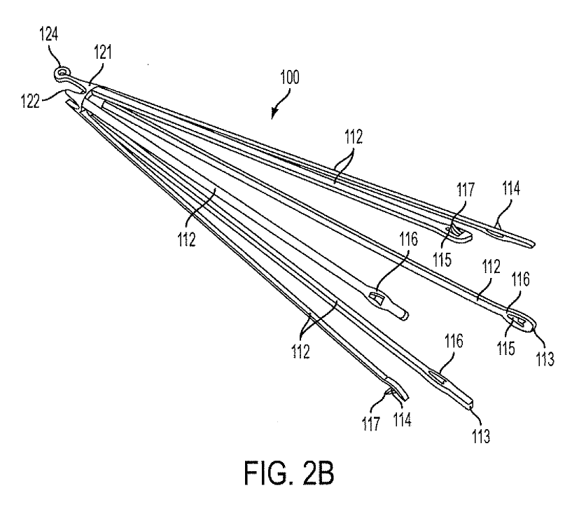

For purposes of explanation and illustration, and not limitation, a partial view of an exemplary embodiment of a vein filter in accordance with the present invention is shown in FIGS. 1A-1C, and is designated generally by reference numeral 10. Other embodiments of a vein filter, or aspects thereof, are provided in FIGS. 2A-23, as will be described. The vein filters of the present invention can be used in a subject's vena cava, or in any other suitable blood vessel, for reducing or preventing harm arising from blood clots.

As used herein, the term distal refers to the portion of a filter further downstream in the direction of blood flow when placed and the term proximal refers to the portion of the filter further upstream. Stated another way, when inserted in a femoral approach to the vena cava, distal refers to the portion further from the user and proximal refers to the portion closer to the user. (In a jugular or upper body approach, the distal portion of the filter is closer to the user.) Thus, in the filters disclosed herein, a retrieval hook is in the distal region of the filter and the hooks, barbs, or other anchoring means which engage the vessel wall to retain the filter are in the proximal region of the filter.

Filter 10 is preferably formed as a continuous, integral structure from a tube. The tube is preferably laser cut and can be formed from a material such as stainless steel, titanium or other suitable material(s) with sufficient springiness so that the legs 12 of the filter are biased to the spread apart position of FIG. 1A. It is also contemplated that filter 10 can be formed from a laser cut tube of shape memory material, such as Nitinol or Elgiloy® alloy available from Elgiloy Specialty Metals of Elgin, Ill., with a shape memorized position shown in FIG. 1A. The filter can have optionally have an anti-proliferative coating, such as Dexamethasone or suitable other coating.

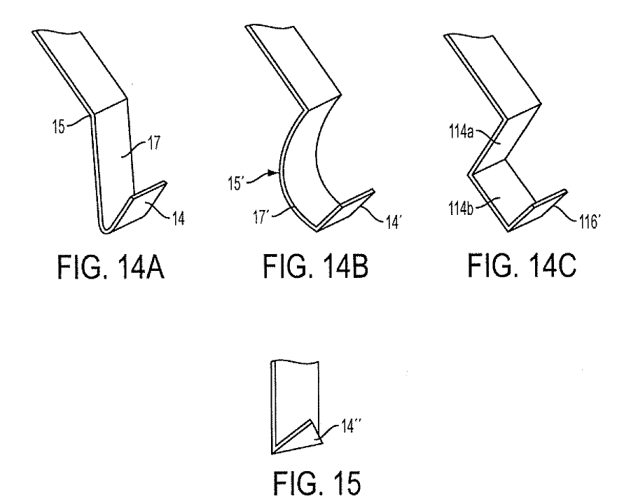

As shown, six legs or struts 12 are formed, and are spaced substantially equally apart circumferentially at about 60 degree increments, although fewer or greater number of legs and different spacing with respect to each other is contemplated. The legs 12 terminate in outwardly extending hooks 14 which engage the vessel wall. (For clarity not all the identical parts are labelled in each of the drawings). Each of the struts or legs 12 bends radially outwardly, with a slight curve, to region 15, where it extends in a substantially longitudinal direction (see also FIG. 14A). This substantially linear portion of legs 12 is designated by reference numeral 17. This bend reduces the profile of the filter in the collapsed condition as it keeps the hooks 14 more in alignment with the other regions of the legs 12. Note that in another embodiment, instead of being substantially straight, the region 15 can curve inwardly as shown in FIG. 14B where region (portion) 15′ is radiused. The radius (i.e. region 15′ in FIG. 14b) can be varied in other embodiments. This inwardly directed arcuate region 17′ transitions into hook 14′ extending radially outward. As another possibility, the region can transition via transverse walls 114a, 114b of FIG. 14C, transitioning into radially outward extending hook 116′. The wall 114b is substantially perpendicular to wall 114a and hook 116′ as shown. Also, the hooks are shown in the embodiment of FIGS. 1A-1C with blunt squared off tips; however, the hooks can have sharpened tips 14″ as shown for example in FIG. 15.

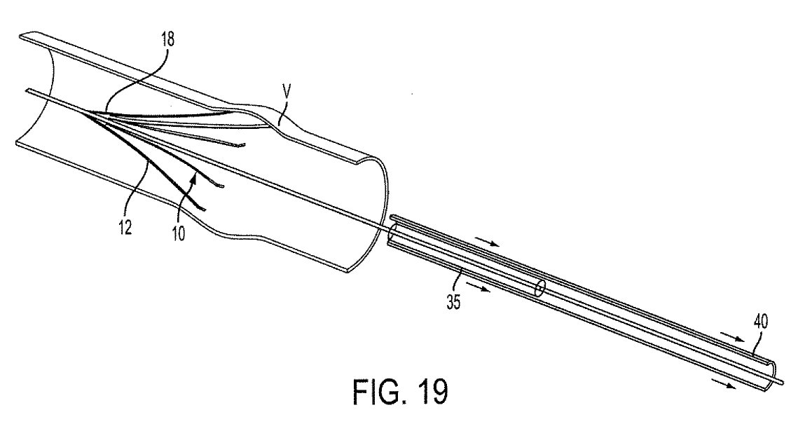

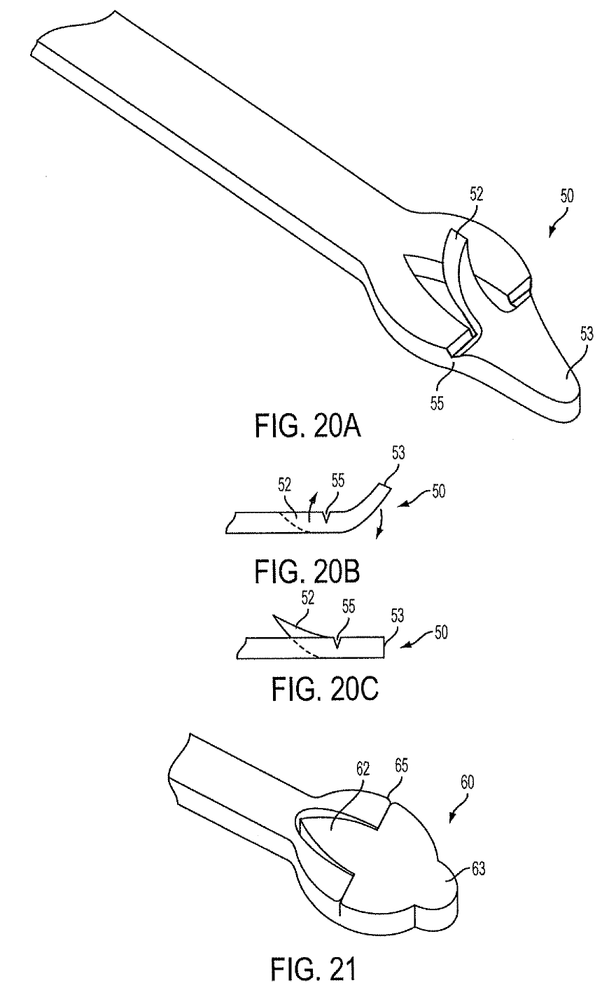

It is also possible to use retractable hooks or barbs at the ends of legs 12, as shown in FIGS. 20A-23. Retractable barb 50 includes a barb 52 connected to a proximal tip 53. Barb 52 and tip 53 are connected to a vein filter leg (e.g. leg 12) by means of living hinge 55. FIG. 20B shows retractable barb 50 in the retracted position, with proximal tip 53 extended so as to contact a blood vessel wall before barb 52. When tip 53 is pressed against a vessel wall, as during deployment of legs 12 into the open position, hinge 55 bends and barb 52 moves into contact with the vessel wall to anchor the vein filter thereto, as indicated in FIG. 20C.

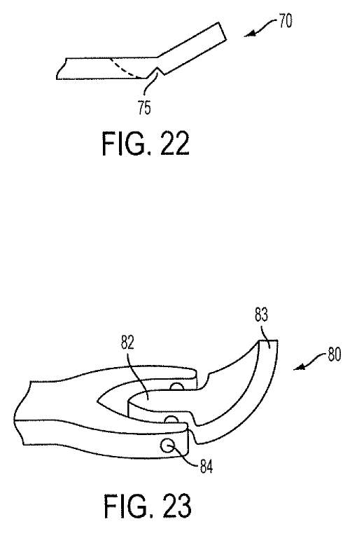

While retractable barb 50 has a proximal tip 53 that is curved outward to face a vessel wall when living hinge 55 is in a relaxed or default position, it is also possible to have the proximal tip 63 substantially flush with a barb 62 and the rest of the vein filter leg, as shown in retractable barb 60 of FIG. 21. Proximal tip 63 is the first portion of the filter leg to contact a vessel wall during deployment, since the leg is angled outward toward the vessel wall during deployment. When tip 63 is pressed into the vessel wall during deployment of the vein filter, living hinge 65 will be bent to extend barb 62, much as described above. While both retractable barbs 50 and 60 include living hinges defined by notches in the outward face of the respective filter legs, it is also possible to provide a living hinge in the inward surface of the filter leg, as in living hinge 75 of retractable barb 70 shown in FIG. 22. Moreover, as shown in FIG. 23, retractable barb 80 can be used without living hinges. Instead, a two piece construction including barb 82, proximal tip 83, and pivot 84 can be hinged to a vein filter leg (e.g. leg 12) having pivot receptacles defined therein. Those skilled in the art will readily appreciate that any suitable hinge means can be used in conjunction with a retractable barb without departing from the scope of the invention.

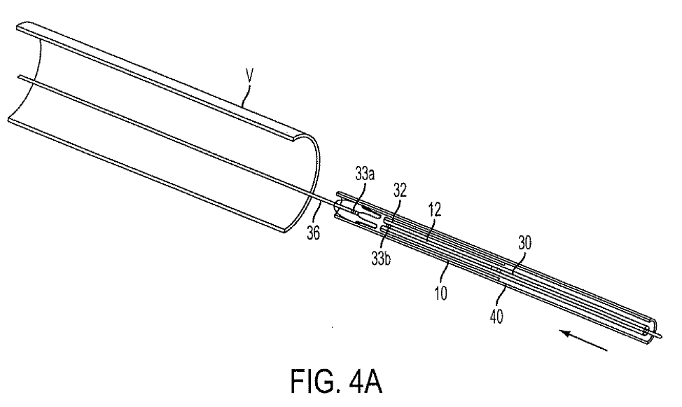

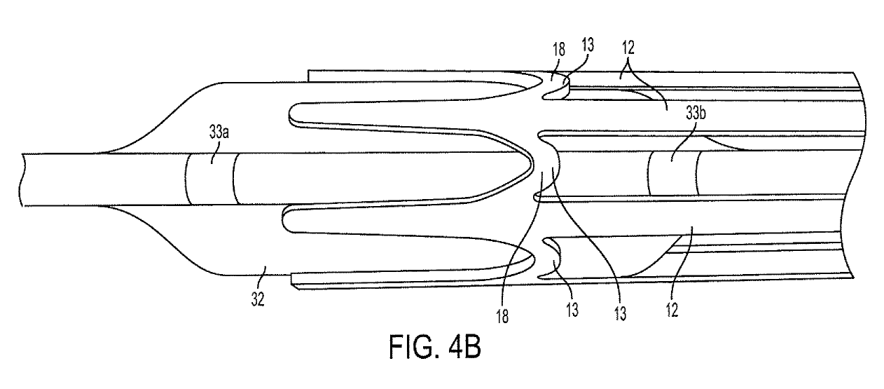

Legs 12 are spaced apart at their distal most end, forming a receptacle or circular gap 16 for receipt of a balloon catheter (and guidewire) described in more detail below. Slightly proximal of the distal most end 19 of the legs 12, the struts are joined by arcuate connecting regions 18. These connecting regions 18 are U-shaped with curved apices 13 (see also FIG. 4B) and provide bridging structure that defines a pivot point for each leg 12 between proximal and distal portions thereof so that inflation of the balloon will cause the proximal portions of legs 12 to pivot inwardly toward a reduced diameter lower profile position.

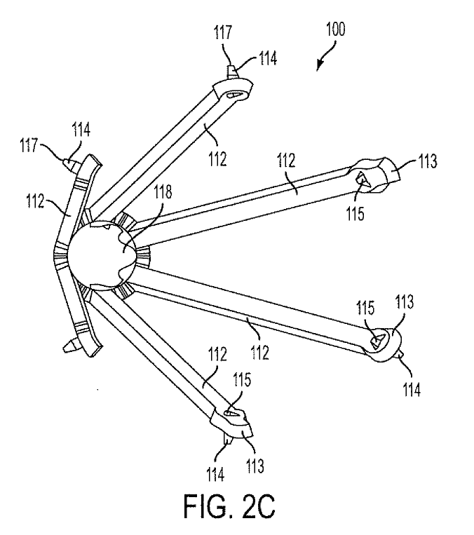

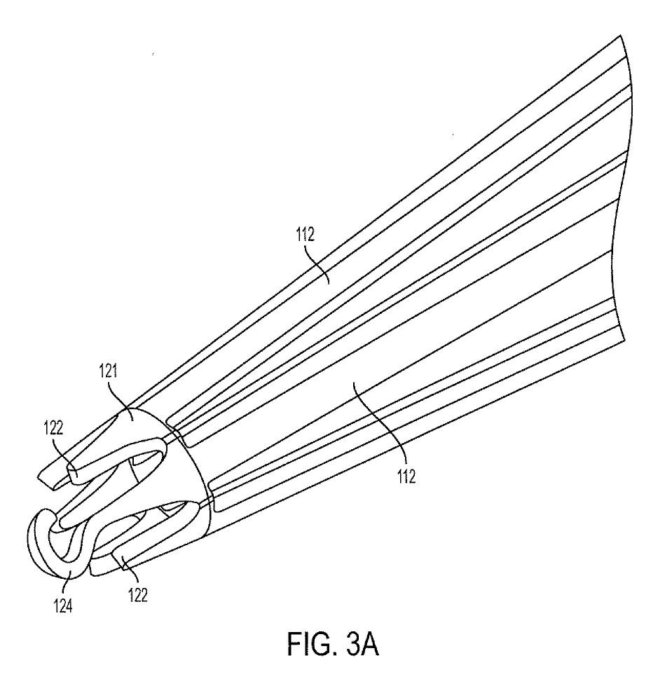

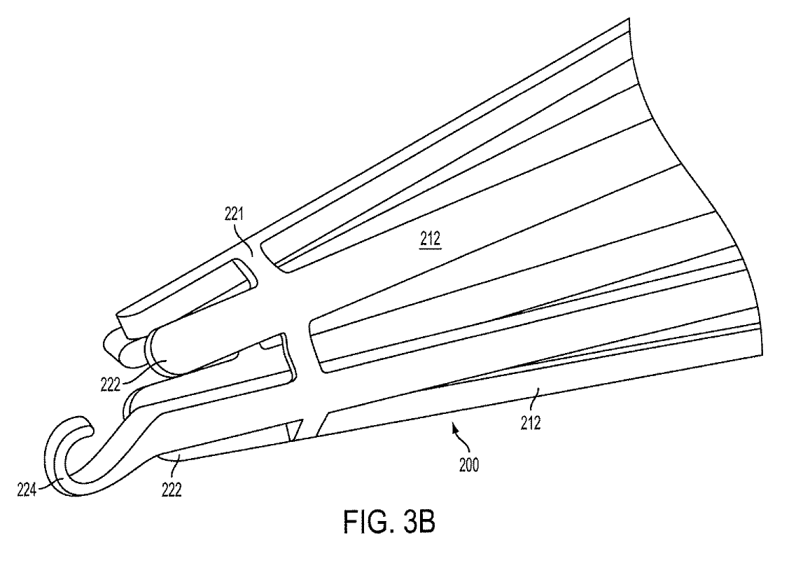

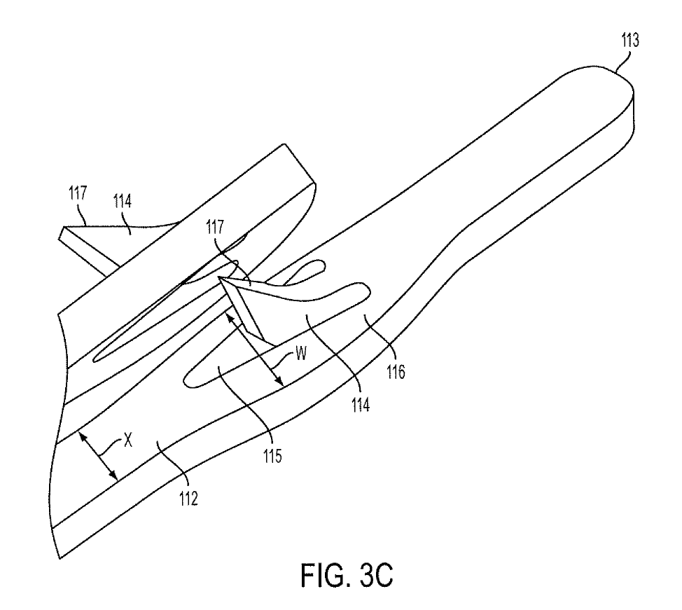

With reference now to FIGS. 2A-3C, another embodiment of a filter designated generally by reference numeral 100 is depicted. Like filter 10, filter 100 is preferably formed from a laser cut tube which can be formed from a material such as stainless steel, titanium or other material(s) with sufficient springiness so that the legs 112 of filter 100 are biased to the spread apart position of FIG. 2B. Filter 100 can also be formed from a laser cut tube of shape memory material, such as Nitinol or Elgiloy®, with a shape memorized position shown in FIG. 2B. Note the shape memory can be set to a filter diameter larger or smaller than that shown in FIG. 2B. As with the embodiment of FIG. 1A, the extent of movement to the deployed position will vary with the internal diameter of the vessel in which the filter is placed.

As shown, six legs or struts 112 are formed, preferably spaced about 60 degrees apart circumferentially, although fewer or greater number of legs and different spacing with respect to each other is contemplated. Legs 112 terminate in blunt rounded ends 113. Referring to FIG. 3C, spaced slightly distally of each of the blunt ends 113 is a hook or barb 114 for engaging the vessel wall. The hooks 114 are spaced from the proximal most ends 113 of the filter legs 112 to reduce the possibility of excessive filter penetration through the vessel wall. In the embodiment shown, the hook 114 is formed by the cut-out 115 (which can be formed during manufacture in the surface of the legs 112) and extends radially outward from the cut-out region to point in a direction away from the longitudinal axis of the filter. For clarity not all the identical parts are labelled in each of the drawings. The hooks 114 preferably have triangular shaped pointed tips 117, best shown in FIG. 3C, however other shapes and tips are also contemplated. While described herein as hooks or barbs 114, those skilled in the art will readily appreciate that any other shapes or forms of hooks can be used without departure form the scope of the invention.

Each of the legs or struts 112 extends radially outwardly, along a substantially linear path, at an angle to the longitudinal axis of filter 100. A wider region 116 of struts 112, having a width w greater than the width x of the other portion of the strut 112 (see FIG. 3C) is provided in the region from which the hooks 114 extend. This provides increased area to form hooks 114 and adds to the structural rigidity of the struts 112. Widened or enlarged region 116 also provides an increased surface area to reduce the possibility of filter penetration through the vessel wall.

The widened regions 116 are preferably substantially teardrop shaped and are preferably staggered so that adjacent regions 116 are longitudinally offset from each other to facilitate collapse of the filter 100 to a lower profile for delivery. Adjacent hooks 114 are likewise preferably longitudinally offset as shown in FIG. 2A.

Legs or struts 112, like legs 12 of the embodiment of FIG. 1A, are spaced apart at their distal most end, forming a circular gap or opening 118 (see FIG. 2C) for receipt of a balloon catheter (and guidewire) described in more detail below. Slightly proximal of the distal most end of the struts 112, the struts are joined by connecting region 121 (see also the close up view of FIG. 3A), which provides bridging structure between legs 112 and provides the pivot points for legs 112 so that like filter 10 of FIG. 1A, inflation of the balloon against the distal leg portions will cause the proximal portions of legs 112 to pivot inwardly toward a reduced diameter lower profile position. Each of the legs 112 terminates distally in a blunt end 122. One of the legs 112 terminates in a retrieval hook 124 (FIG. 3A) configured to receive a conventional snare or other retrieval device to enable the filter 100 to be pulled in a distal direction for removal. Hook 124 is off center to maintain a sufficient gap for insertion of the balloon (described below). Those skilled in the art will readily appreciate that hook 124 is optional. Moreover, any other suitable retrieval means can also be used in lieu of hook 124 without departing from the scope of the invention.

FIG. 3B shows a distal portion of vein filter 200 showing another possible bridging structure configuration. Legs 212 are joined near their distal ends 222 by bridging structure 221 that is generally straight between respective adjacent legs 212, which are otherwise substantially uniform in width across the bridging structure. One of the distal leg portions terminates in a retrieval hook 224 which functions as described above for retrieval.

Turning now to the methods of use of a filter in accordance with the present invention, and with reference to FIGS. 4A-13, filter 10 is shown and described by way of example, it being understood that filters 100/200 can be used in a similar fashion. Filter 10 is inserted into the vessel V as shown in FIG. 4A (see also FIG. 4B, filter 10 is partially cut away in some of the views for clarity). A balloon catheter 30 (shown truncated in some views for clarity) with a balloon 32 in the inflated condition is inserted over guidewire 36 through the space between the legs 12 and into the gap 16 (gap 16 is shown in FIGS. 1A-1C). Note the catheter 30 (and/or balloon) can optionally have markers such as bands or rings 33a, 33b, or any other suitable means for use in imaging such as in fluoroscopy. Marker bands 33a/33b also delineate the region for placement of the balloon so the center of the balloon, which has the largest diameter, will be positioned just distal of the pivot point at the distal end of the legs 12 as shown in FIG. 4B. Since balloon 32 is inflated during delivery, it pivots the legs 12 to the first or reduced profile position. The filter 10 and balloon catheter 30 are shown in FIG. 4A within delivery sheath 40 (a portion of which is cut away for clarity), which facilitates placement of filter 10 within the blood vessel.

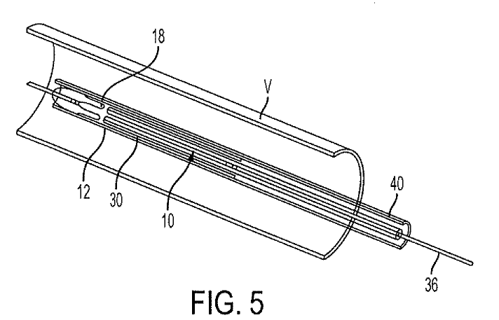

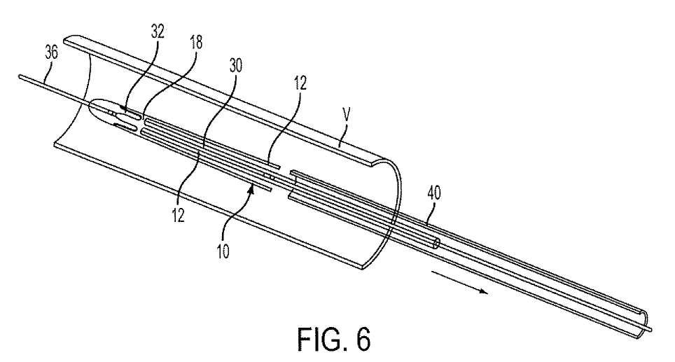

After insertion of sheath 40 and filter 10 to the desired position of the vessel V (FIG. 5), delivery sheath 40 is withdrawn to expose the filter 10 as indicated by the arrow in FIG. 6. Legs 12 may move slightly outwardly since they may no longer be constrained by the inner wall of the sheath 40, but remain substantially collapsed due to the presence of the inflated balloon 32. Note the diameter of the balloon can be varied to vary the position of legs 12 (and hooks 14) in the collapsed insertion position. That is, for example, if a balloon of sufficiently large diameter is utilized, it can pivot the legs sufficiently inwardly so they are biased against the balloon catheter. In such instance, removal of the sheath would not affect movement of the legs. In other variations using a smaller diameter balloon, the legs 12 may be pivoted inwardly to a smaller extent so they press against the sheath in the insertion position and move slightly outwardly when the sheath is removed. It is contemplated that in the insertion position, the legs can be biased inwardly a sufficient distance so that the hooks do not exceed the outer diameter of the collapsed filter, thus minimizing interference with the sheath. Maintaining the hooks in this low profile position (within the outer diameter of the other portions of the filter) is also achieved in part by the inwardly bent region 15 (shown in FIG. 14A).

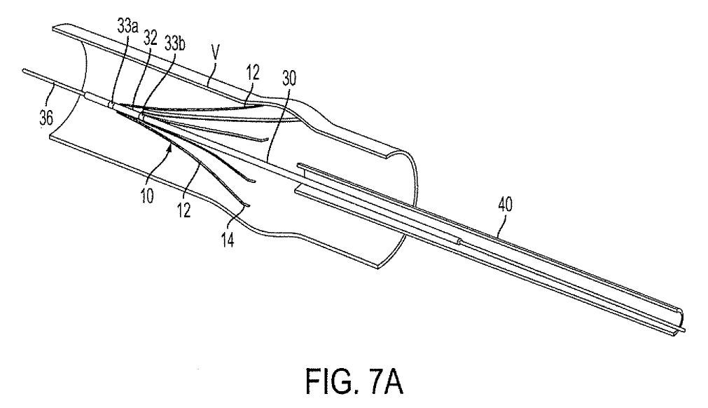

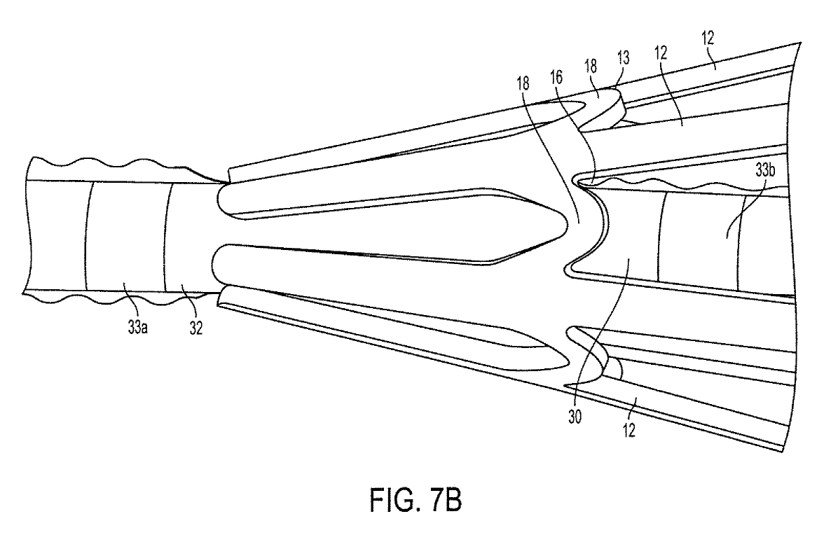

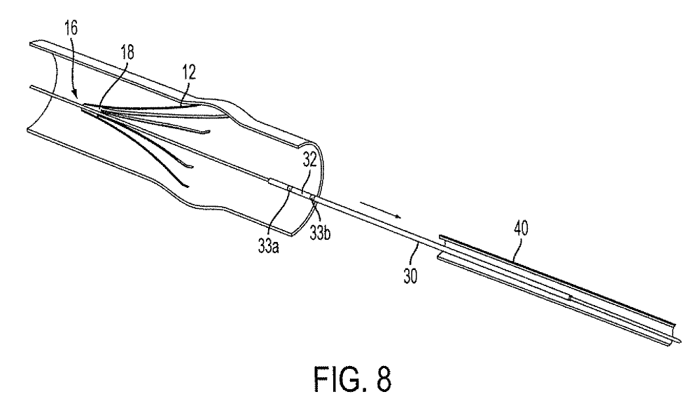

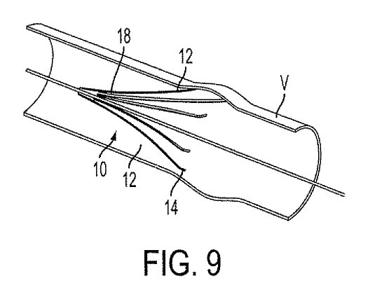

With reference to FIG. 7A, after removal of the sheath, balloon 32 can be deflated, causing the legs 12 to spring or expand outwardly as shown in FIGS. 7A and 7B. (If shape memory is utilized, balloon deflation will enable the legs to move to their shape memorized position). As shown, hooks 14 will engage the internal vessel wall. The balloon catheter 30, delivery sheath 40 and guidewire 36 are then removed (as indicated by the arrow in FIG. 8), leaving the filter 10 in place as shown in FIG. 9.

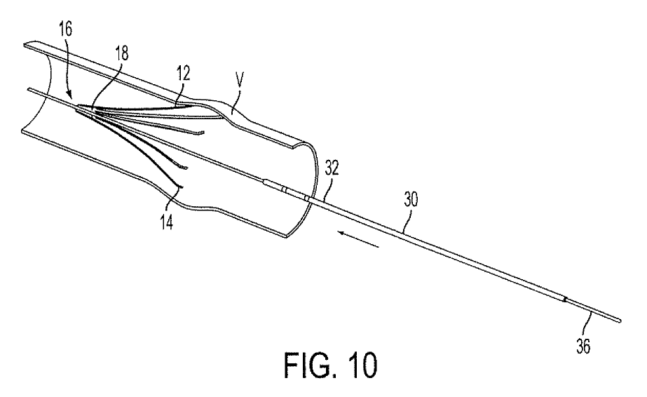

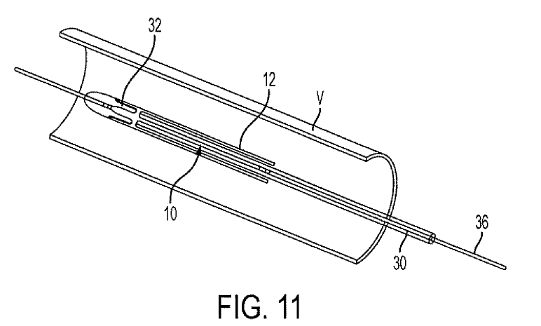

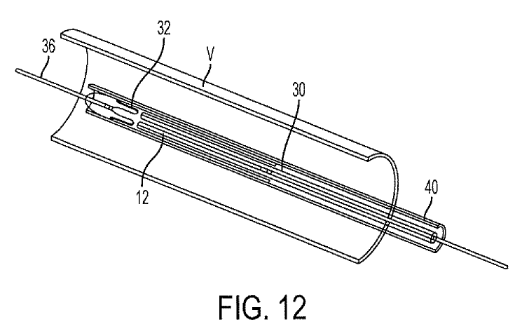

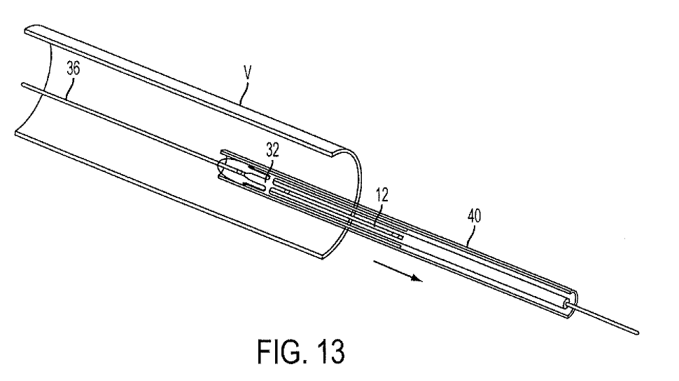

If desired, the vessel filter 10 (or filters 100/200) can be removed from the vein after a period of time, and advantageously, if desired, with the same device used for insertion. The steps of one method of filter removal are illustrated in FIGS. 10-13. The balloon catheter 30 is reinserted over the guidewire 36 to the vessel filter site and into the gap 16 of filter 10 so the center of the balloon 32 is aligned distal of the pivot point. The balloon 32 is then inflated, causing the legs 12 to pivot to the collapsed condition of FIG. 11 (see also FIG. 4B). The delivery sheath 40 can then be slid distally over the filter 10 and balloon catheter 30 as shown in FIG. 12 (or alternatively the filter can be retracted to within the confines of the delivery sheath 40) and then the entire assembly can be removed in the direction of the arrow of FIG. 13. Those skilled in the art will readily appreciate that the description of vein filters herein with use of a catheter balloon is exemplary and that any expandable mechanism or hydraulic, pneumatic, electrical, or other suitable type of expandable member can also be used without departing from the scope of the invention.

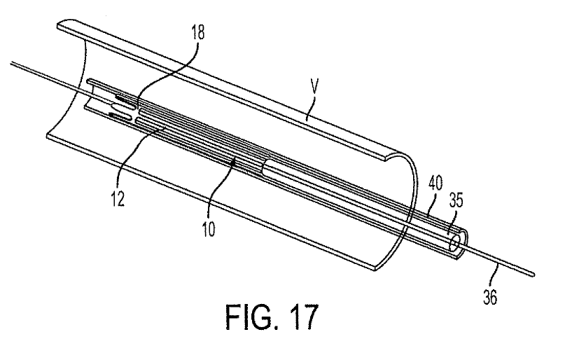

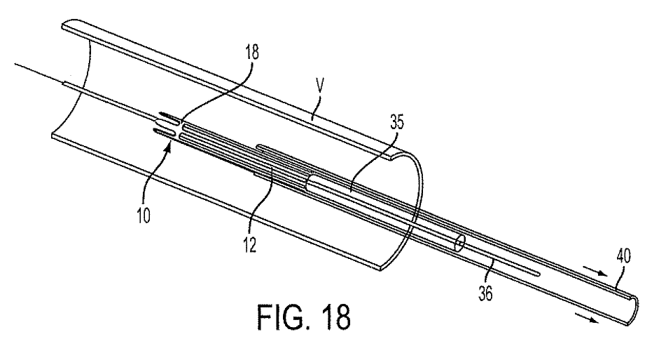

Although the balloon is described for use during initial placement of the filter, it is also contemplated that the filter can be inserted over the guidewire and constrained in a lower profile position within the sheath during delivery without the use of the balloon, as shown in FIG. 17. FIG. 18 shows delivery sheath 40 partially withdrawn while vein filter 10 is held in place at the desired location by pusher member 35 (shown truncated for clarity). Once removed from sheath 40, legs 12 move outwardly toward their second or expanded position, as shown in FIG. 19. If it is desired after initial placement of the filter to move it to another location (or remove it), the balloon can be inserted over the already placed guidewire and inflated to collapse the filter into the first or retracted position described above. With legs 12 in the collapsed position, vein filter 110 can be repositioned as needed. After repositioning to the desired site, the balloon can then be deflated, allowing the legs to move outwardly so the hooks engage the vessel wall.

It is also possible to remove a vein filter 10/100/200 from within a vein without using a balloon catheter. The filter can be removed by insertion of a retrieval snare (e.g. through a jugular approach if the filter is located in the inferior vena cava) which loops around the hook 124 (see FIG. 3A) and is then pulled in a distal direction while a retrieval sheath is moved in a proximal direction to encapsulate and close the legs to dislodge the hooks 114 from the vessel wall. The snare pulls the filter 100 into a retrieval sheath, or holds the filter substantially in place as a retrieval sheath advances over the filter, where the legs 112 are collapsed by the sheath.

It should also be appreciated that as noted above, regardless of whether a balloon catheter is used to deploy a vein filter, the balloon can be utilized to reposition the filter, which is advantageous when interoperative repositioning is warranted. That is, the balloon can be reinserted as discussed above and inflated to move the legs inwardly to release them from engagement with the vessel, enabling the filter to be moved with the catheter to another location. As can be appreciated, the filter can be repositioned multiple times, and this repositioning can be achieved, if desired, with the same delivery system used for delivery and removal. Also, the repositioning can be achieved utilizing the balloon catheter without having to pull the filter into a sheath.

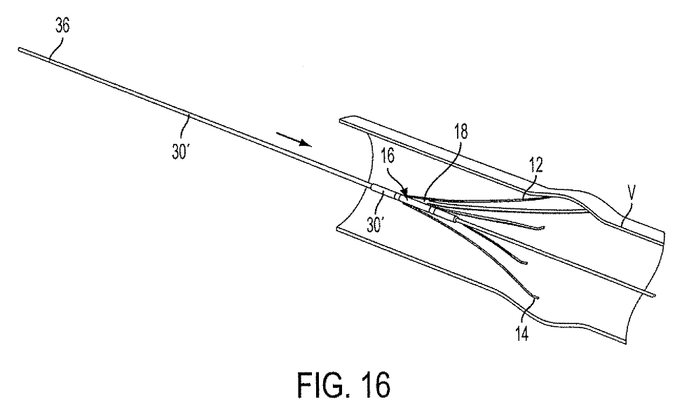

Insertion and repositioning/removal of the filter are described above and illustrated utilizing a femoral approach. It is also contemplated that the filter can be delivered and or repositioned/removed via an approach from the upper part of the body such as the jugular vein, as indicated in FIG. 16. In this approach, the catheter approaches the vessel site from the opposite direction of FIG. 4A, (e.g. see arrow of FIG. 16) and the filter can be loaded in the delivery sheath with the legs facing the delivery opening of the sheath so that the legs can be exposed first, followed by exposure of the pivot region of the filter. To access the filter for repositioning or retrieval, the balloon catheter 30′ can be inserted as shown in FIG. 16 (in the opposite direction from that shown in FIG. 10) with the balloon placed adjacent the pivot region.

Whether or not a balloon catheter is used for initial placement and removal, the filter devices and methods described above provide significant advantages because of the ease with which a balloon catheter can be used to reposition the filter if needed. As described above typical vein filters cannot be easily repositioned if initially misplaced within a vein. Misplacement of typical vein filters is frequent and if severe enough, can result in extra costs for replacement of the vein filter to achieve proper placement. The filter devices and methods of the present invention are particularly advantageous over previously known devices and methods since, among other things, the filter devices and methods of the present invention allow for easy repositioning of initially misplaced vein filters without the need to replace the filter.

While the description above contains exemplary specifics, those specifics should not be construed as limitations on the scope of the disclosure, but merely as exemplifications or preferred embodiments thereof. For example, the filter can be inserted in other regions of the body besides the inferior vena cava. Those skilled in the art will envision many other possible variations that are within the scope and spirit of the disclosure as defined by the claims appended hereto. Thus, while the apparatus and methods of the subject invention have been shown and described with reference to preferred embodiments, those skilled in the art will readily appreciate that changes and/or modifications may be made thereto without departing from the spirit and scope of the subject invention.

What is claimed is:

1. A vein filter comprising: a plurality of circumferentially spaced apart elongated legs having opposed proximal and distal leg portions, wherein adjacent distal leg portions are connected to one another by bridging structure and wherein the legs are adapted and configured for movement between a first position wherein the proximal leg portions are substantially parallel to one another and a second position wherein the proximal leg portions are circumferentially divergent from one another to anchor the filter within a blood vessel.

2. A vein filter as recited in claim 1, wherein the legs and bridging structure are biased to the second position to anchor the filter within a blood vessel.

3. A vein filter as recited in claim 2, wherein the legs and bridging structure include a shape memory material biasing the legs and bridging structure to the second position.

4. A vein filter as recited in claim 2, wherein the distal leg portions define a receptacle configured and adapted to receive an expanding member for expanding against the distal leg portions to place the legs in the first position, and for contracting to allow the distal leg portions to converge to place the legs in the second position to anchor the filter within a blood vessel.

5. A vein filter as recited in claim 1, wherein each of the proximal leg portions includes means for anchoring the filter to a blood vessel wall.

6. A vein filter as recited in claim 1, wherein each of the proximal leg portions includes an enlarged section configured and adapted to engage a blood vessel wall to limit penetration thereof.

7. A vein filter as recited in claim 1, wherein the legs are spaced substantially equally apart circumferentially.

8. A vein filter as recited in claim 1, wherein adjacent distal leg portions are connected to one another by bridging structure that defines a pivot point for movement of each of the legs between the first and second positions.

9. A vein filter as recited in claim 8, wherein the legs and bridging structure are a continuous integral structure.

10. A vein filter as recited in claim 1, further comprising retrieval means connected to at least one distal leg portion, the retrieval means being configured and adapted to receive a retrieval device for moving the vein filter distally.

11. A kit for placing a vein filter within a vein comprising:
a) a vein filter including a plurality of circumferentially spaced apart elongated legs having opposed proximal and distal leg portions, wherein adjacent distal leg portions are connected to one another by bridging structure and wherein the legs are adapted and configured for movement between a first position wherein the proximal leg portions are substantially parallel to one another and a second position wherein the proximal leg portions are circumferentially divergent from one another to anchor the filter within a blood vessel, and wherein the distal leg portions define a receptacle configured and adapted to receive a catheter balloon;
b) a catheter balloon configured and adapted to expand against the distal leg portions to place the legs of the filter in the first position, and to contract to allow the distal leg portions to converge to place the legs in the second position to anchor the filter within a blood vessel; and
c) a delivery sheath configured and adapted to house the vein filter during placement of the vein filter within a blood vessel.

12. A kit for placing a vein filter as recited in claim 11, wherein the legs of the vein filter are biased to the second position to anchor the filter within a blood vessel.

13. A kit for placing a vein filter as recited in claim 11, wherein each of the proximal leg portions of the vein filter includes means for anchoring the filter to a blood vessel wall.

14. A kit for placing a vein filter as recited in claim 11, wherein the legs of the vein filter are dimensioned and adapted for placement within an inferior vena cava.

15. A kit for placing a vein filter as recited in claim 11, wherein the plurality of legs includes six legs that are equally spaced apart circumferentially.

16. A kit for placing a vein filter as recited in claim 11, wherein the catheter balloon includes means for fluoroscopically determining the position of the of the catheter balloon with respect to the distal leg portions of the vein filter.

17. A method of placing a vein filter within a vein comprising steps of:
a) providing a vein filter including a plurality of circumferentially spaced apart elongated legs having opposed proximal and distal leg portions, wherein adjacent distal leg portions are connected to one another by bridging structure and wherein the legs are adapted and configured for movement between a first position wherein the proximal leg portions are substantially parallel to one another and a second position wherein the proximal leg portions are circumferentially divergent from one another to anchor the filter within a blood vessel, wherein the distal leg portions define a receptacle for receiving a catheter balloon for inflating against the distal leg portions of the vein filter to move the legs into the first position, and wherein the vein filter is housed within a delivery sheath with the legs in substantially the first position for placement within a blood vessel;
b) positioning a guidewire within a vein for guiding the vein filter and delivery sheath to a predetermined location within a vein;
c) guiding the vein filter and delivery sheath into the predetermined location along the guidewire; and
d) withdrawing the delivery sheath from the vein filter to anchor the vein filter within the vein with the legs in the second position.

18. A method of placing a vein filter within a vein as recited in claim 17, wherein the step of guiding includes using fluoroscopy to guide the vein filter to the predetermined location within the vein.

19. A method of placing a vein filter within a vein as recited in claim 17, further comprising a step of repositioning the vein filter by placing a deflated catheter balloon within the receptacle of the distal leg portions of the vein filter, inflating the catheter balloon to place the legs of the vein filter into substantially the first position, relocating the vein filter together with the catheter balloon, deflating the catheter balloon to place the legs of the vein filter into the second position, and removing the catheter balloon from the vein filter.

20. A method of placing a vein filter within a vein as recited in claim 19, wherein the step of repositioning includes placing the deflated catheter balloon within the receptacle of the vein filter by introducing the catheter balloon through a jugular vein.

21. A method of placing a vein filter within a vein as recited in claim 17, wherein the step of providing a vein filter includes providing a catheter balloon within the distal leg portions of the vein filter, the catheter balloon being configured and adapted to expand against the distal leg portions of the vein filter to place the legs of the vein filter in the first position and to contract to place the legs of the vein filter in the second position, wherein the step of withdrawing the delivery sheath from the vein filter includes contracting the catheter balloon to place the legs of the vein filter in the second position to anchor the vein filter within the vein.
